(19) United States
(12) Patent Application Publication
(10) Pub. No.: US 2006/0263360 A1
(43) Pub. Date: Nov. 23, 2006

(54) Compositions and methods for delivering thymosin beta 4, analogues, isoforms and other derivatives

(75) Inventor: Allan L. Goldstein, Washington, DC (US)

Correspondence Address: ROTHWELL, FIGG, ERNST & MANBECK, P.C., 1425 K STREET, N.W., SUITE 800, WASHINGTON, DC, 20005, US

(73) Assignee: RegeneRx BioPharmaceuticals, Inc., Bethesda, MD (US)

(21) Appl. No.: 10/551,348

(22) PCT Filed: Mar. 31, 2004
(86) PCT No.: PCT/US04/09614

§ 371 Date: Jul. 17, 2006

Related U.S. Application Data
(60) Provisional application No. 60/458,399, filed on Mar. 31, 2003.

(51) Int. Cl. A61K 39/395; A61K 38/37; A61K 38/08

(52) U.S. Cl. 424/145.1; 514/12; 514/16; 514/17

(57) ABSTRACT

A composition and method utilizes a substantially purified composition including an adhesive and a polypeptide containing amino acid sequence LKKTET or a conservative variant thereof.

### CROSS-REFERENCE TO RELATED APPLICATION

This application is a National Phase of International Application Serial No. PCT/US2004/009614, filed Mar. 31, 2004, which also claims the benefit of U.S. Provisional Application No. 60/458,399, filed Mar. 31, 2003.

### BACKGROUND OF THE INVENTION

1. Field of the Invention

The present invention relates to the field of compositions and methods for delivering polypeptide pharmaceuticals.

2. Description of the Background Art

Polypeptide pharmaceuticals can be extremely efficacious agents in the treatment of various maladies. Since polypeptide pharmaceuticals can be very expensive to produce, there is a need in the art for improved compositions and methods for delivering polypeptide pharmaceuticals.

### SUMMARY OF THE INVENTION

In accordance with the present invention, a composition comprises a substantially purified composition including an adhesive and a polypeptide comprising amino acid sequence LKKTET SEQ ID NO: 1, or a conservative variant thereof. A method of delivery of a polypeptide to a site comprises introducing the above composition to the site.

### DETAILED DESCRIPTION OF THE INVENTION

The present invention provides compositions and methods utilizing actin-sequestering peptides such as thymosin β4 (Tβ4) and other actin-sequestering peptides or peptide fragments containing amino acid sequence LKKTET SEQ ID NO: 1 or conservative variants thereof. Included are N- or C-terminal variants such as KLKKTET SEQ ID NO:2 and LKKTETQ SEQ ID NO:3. These peptides and peptide fragments are useful in promoting wound healing and other physiological uses.

Thymosin β4 was initially identified as a protein that is up-regulated during endothelial cell migration and differentiation in vitro. Thymosin β4 is a 43 amino acid, 4.9 kDa ubiquitous polypeptide identified in a variety of tissues. Several roles have been ascribed to this protein including a role in a endothelial cell differentiation and migration, T cell differentiation, actin sequestration and vascularization.

Thymosin β4 is a member of the β-thymosin family of highly conserved polar 5-kDa polypeptides found in various tissues and cell types. Originally purified from thymus and regarded as a thymic hormone, thymosin β4 was then found to be involved in multiple biological processes. As the main G-actin sequestering peptide, it plays an important role in regulation of actin assembly during cell proliferation, migration, and differentiation. Numerous studies implicate thymosin β4 in regulation of cancerogenesis, inflammation, angiogenesis, and wound healing. It was found that thymosin β4 expression regulated tumorigenicity and metastatic activity in malignant cell lines through actin-based cytoskeletal organization. Thymosin β4 was found to be elevated in tube forming endothelial cells; it increases their attachment, spreading and migration thus promoting angiogenesis. Thymosin β4 was also found in ulcer extracts and wound fluids at high concentrations and was suggested to function as an antibacterial factor. The stimulating role of thymosin β4 in wound healing was demonstrated in several studies with animal models. When added topically or administered intraperitoneally, thymosin β4 enhanced dermal wound healing in a rat full thickness model. The ability to accelerate dermal wound healing has also been observed in db/db diabetic mice, steroid-immunosuppressed mice and in aged mice. Thymosin β4 has also been shown to accelerate healing of the corneal epithelium after burn injuries and to down regulate a number of corneal cytokines and chemokines reducing the inflammatory response.

Activation of the coagulation cascade upon vascular injury results in generation of thrombin which converts fibrinogen into fibrin. Fibrin polymerizes spontaneously to form blood clots which seals damaged places thus preventing the loss of blood. Fibrin also serves as a provisional matrix on which various cell types adhere, migrate and proliferate replacing fibrin with normal tissues during subsequent wound healing processes. Factor XIIIa, a plasma transglutaminase, covalently cross-links the fibrin clot reinforcing its structure. In addition, it also cross-links to fibrin a number of physiologically active proteins which may modulate properties of the fibrin matrix. For example, covalent incorporation of α₂-antiplasmin increases resistance of the matrix to fibrinolysis and incorporation of fibronectin may affect its ability to support cell adhesion and migration. Tissue transglutaminase can selectively incorporate into fibrin thymosin β4.

Thymosin β4 serves as a specific substrate for tissue transglutaminase and can be selectively cross-linked by it to collagen, actin, fibrinogen and fibrin, proteins which are also involved in the above mentioned processes. After activation of platelets with thrombin, thymosin β4 is released and cross-linked to fibrin in a time- and calcium-dependent manner. Platelet factor XIIIa is co-released from stimulated platelets. Cross-linking of platelet-released thymosin β4 to fibrin appears to be mediated by factor XIIIa and provides a mechanism to increase the local concentration of thymosin β4 near sites of clots and tissue damage, for promotion of wound healing, angiogenesis and inflammatory response.

Fibrinogen is a chemical dimer comprising two identical subunits, each composed of three polypeptide chains, Aα, Bβ and γ held together by a number of disulfide bonds. The disulfide-linked NH₂-terminal portions of all six chains form the central E region, while the COOH-terminal portions form two terminal D regions and two αC-domains. Upon conversion of fibrinogen into fibrin, thrombin-mediated removal of the NH₂-terminal fibrinopeptides A and B from the fibrinogen and removal of the NH₂-terminal fibrinopeptides A and B from the fibrinogen Aα and Bβ chains, respectively, results in exposure of their active sequences (polymerization sites) and enables interaction between the E and D regions of neighboring molecules (DD:E interaction) to form a fibrin polymer. The polymer becomes cross-linked by factor XIIIa through the COOH-terminal portions of the fibrin α and γ chains. The intermolecular cross-linking of the γ chains of the adjacent D regions occurs rapidly resulting in γ-γ dimers, while cross-lining between the α polymers (αC-domains) occurs more slowly and results in formation of α polymers. In addition, the α chains serve for cross-linking to fibrin of such proteins as fibronectin, α₂-antiplasmin, and PAI-2. Thus, it is tempting to hypothesize that these chains could also be involved in cross-linking of thymosin β4.

To clarify the mechanism of the incorporation of thymosin β4 into fibrin(ogen), its interaction was studied with fibrinogen, fibrin and their recombinant fragments (domains) in the absence and presence of factor XIIIa. The study revealed that although there appears to be no substantial non-covalent interaction between fibrin(ogen) and thymosin β4, factor XIIIa efficiently cross-links the latter to both fibrinogen and fibrin and that cross-liking occurs mainly through the COOH-terminal portion of their αC-domains including residues 392-610.

In accordance with one embodiment, a substantially purified composition is provided which includes an adhesive and a polypeptide comprising amino acid sequence LKKTET SEQ ID NO: 1 or a conservative variant thereof. In accordance with one embodiment, the adhesive is capable of adhering to medical devices such as stents. In a particularly preferred embodiment, the adhesive is capable of adhering to tissues of a living subject such as a human.

In preferred embodiments, the adhesive is a biodegradable adhesive. When used herein, the term biodegradable adhesive is intended to encompass bioabsorbable or errodable adhesives. In preferred embodiments, the invented composition initially is in a fluid or semi-fluid state, most preferably in a liquid or semi-liquid state. In particularly preferred embodiments, after application, the adhesive increases in viscosity or at least partially solidifies while adhering to the tissue. The composition may be introduced by applying to an area in a layer, most preferably by spraying or with a brush.

In preferred embodiments, the adhesive utilized in the present invention is a fibrin sealant matrix (fibrin glue). Fibrin glue is a two-component system of separate solutions of fibrinogen and thrombin/calcium. When the two solutions are combined, the resultant mixture mimics the final stages of the clotting cascade to form a fibrin clot. The fibrinogen component can be prepared extemporaneously from autologous, single-donor, or pooled blood. Fibrin glue is available in Europe under the brand names Beriplast, Tissel, and Tissucol. Fibrin glue has been used in a wide variety of surgical procedures to repair, seal, and attach tissues in a variety of anatomic sites.

Thus, the present invention provides a method of delivering an LKKTET SEQ ID NO: 1 polypeptide to a site of a living subject. In preferred embodiments, this site is a surface. The inventive method comprises applying the inventive composition to the site. In preferred embodiments, the site is a wound, such as an acute or chronic wound.

In preferred embodiments, the adhesive is fibrin, fibrinogen, fibrin glue, a collagen, fragments of any of the above or a mixture of any of the above. Collagen adhesives which may be utilized include types 1, 2, 3, 4 and/or 5 collagens. Other adhesives may include actin or integrin adhesives.

In other embodiments, the biodegradable adhesive utilized in the inventive composition is a gel (e.g., adhesive collagen gel), gel/fibrin mixture, powder or the like.

In preferred embodiments, the adhesive is covalently bound to the SEQ ID NO: 1 peptide, most preferably by factor XIIIa. In particularly preferred embodiments, the adhesive is a fragment of fibrin or fibrinogen.

In preferred embodiments, the LKKTET SEQ ID NO: 1 polypeptide comprises amino acid sequence KLKKTET SEQ ID NO:2 or LKKTETQ SEQ ID NO:3, Thymosin β4 (Tβ4), an N-terminal variant of Tβ4, a C-terminal variant of Tβ4, an isoform of Tβ4, a splice-variant of Tβ4, oxidized Tβ4, Tβ4 sulfoxide, lymphoid Tβ4, pegylated Tβ4 or any other actin sequestering or bundling proteins having actin binding domains, or peptide fragments comprising or consisting essentially of the amino acid sequence LKKTET SEQ ID NO: 1 or conservative variants thereof. International Application Serial No. PCT/US99/17282, incorporated herein by reference, discloses isoforms of Tβ4 which may be useful in accordance with the present invention as well as amino acid sequence LKKTET SEQ ID NO: 1 and conservative variants thereof, which may be utilized with the present invention. International Application Serial No. PCT/GB99/00833 (WO 99/49883), incorporated herein by reference, discloses oxidized Thymosin β4 which may be utilized in accordance with the present invention. Although the present invention is described primarily hereinafter with respect to Tβ4 and Tβ4 isoforms, it is to be understood that the following description is intended to be equally applicable to amino acid sequence LKKTET SEQ ID NO: 1, LKKTETQ SEQ ID NO:3, peptides and fragments comprising or consisting essentially of LKKTET SEQ ID NO: 1 or LKKTETQ SEQ ID NO:3, conservative variants thereof, as well as oxidized Thymosin β4.

Examples of contacting the damaged site include contacting the site with a composition comprising adhesive/Tβ4 alone, or in combo with at least one agent that enhances Tβ4 penetration, or delays or slows release of Tβ4 peptides into the area to be treated. A subject may be a mammal, preferably human.

Tβ4, or its analogues, isoforms or derivatives, may be administered in any suitable effective amount. For example, Tβ4 may be administered in dosages within the range of about 0.1-50 micrograms of Tβ4, more preferably in amounts within the range of about 1-25 micrograms.

A composition in accordance with the present invention can be administered daily, every other day, etc., with a single administration or multiple administrations per day of administration, such as applications 2, 3, 4 or more times per day of administration.

Tβ4 isoforms have been identified and have about 70%, or about 75%, or about 80% or more homology to the known amino acid sequence of Tβ4. Such isoforms include, for example, Tβ4^{ala}, Tβ9, Tβ10, Tβ11, Tβ12, Tβ13, Tβ14 and Tβ15. Similar to Tβ4, the Tβ10 and Tβ15 isoforms, as well as the Tβ4 splice-variants, have been shown to sequester actin. Tβ4, Tβ10 and Tβ15, as well as these other isoforms share an amino acid sequence, LKKTET SEQ ID NO: 1, that appears to be involved in mediating actin sequestration or binding. Although not wishing to be bound to any particular theory, the activity of Tβ4 isoforms may be due, in part, to the ability to regulate the polymerization of actin. β-thymosins appear to depolymerize F-actin by sequestering free G-actin. Tβ4's ability to modulate actin polymerization may therefore be due to all, or in part, its ability to bind to or sequester actin via the LKKTET SEQ ID NO: 1 sequence. Thus, as with Tβ4, other proteins which bind or sequester actin, or modulate actin polymerization, including Tβ4 isoforms having the amino acid sequence LKKTET SEQ ID NO: 1, are likely to be effective, alone or in a combination with Tβ4, as set forth herein.

Thus, it is specifically contemplated that known Tβ4 isoforms, such as Tβ4^{ala}, Tβ9, Tβ10, Tβ11, Tβ12, Tβ13, Tβ14 and Tβ15, as well as Tβ4 isoforms and Tβ4 splice-variants not yet identified, will be useful in the methods of the invention. As such Tβ4 isoforms are useful in the methods of the invention, including the methods practiced in a subject. The invention therefore further provides pharmaceutical compositions comprising Tβ4, as well as Tβ4 isoforms Tβ4^{ala}, Tβ9, Tβ10, Tβ11, Tβ12, Tβ13, Tβ14 and Tβ15, and a pharmaceutically acceptable carrier.

In addition, other proteins having actin sequestering or binding capability, or that can mobilize actin or modulate actin polymerization, as demonstrated in an appropriate sequestering, binding, mobilization or polymerization assay, or identified by the presence of an amino acid sequence that mediates actin binding, such as LKKTET SEQ ID NO: 1, for example, can similarly be employed in the methods of the invention. Such proteins include gelsolin, vitamin D binding protein (DBP), profilin, cofilin, adsevertin, propomyosin, fincilin, depactin, DnaseI, villin, fragmin, severin, capping protein, β-actinin and acumentin, for example. As such methods include those practiced in a subject, the invention further provides pharmaceutical compositions comprising gelsolin, vitamin D binding protein (DBP), profilin, cofilin, depactin, DnaseI, villin, fragmin, severin, capping protein, β-actinin and acumentin as set forth herein. Thus, the invention includes the use of a polypeptide comprising the amino acid sequence LKKTET SEQ ID NO: 1 (which may be within its primary amino acid sequence) and conservative variants thereof.

As used herein, the term “conservative variant” or grammatical variations thereof denotes the replacement of an amino acid residue by another, biologically similar residue. Examples of conservative variations include the replacement of a hydrophobic residue such as isoleucine, valine, leucine or methionine for another, the replacement of a polar residue for another, such as the substitution of arginine for lysine, glutamic for aspartic acids, or glutamine for asparagine, and the like.

Tβ4 has been localized to a number of tissue and cell types and thus, agents which stimulate the production of Tβ4 can be added to or comprise a composition to effect Tβ4 production from a tissue and/or a cell. Such agents include members of the family of growth factors, such as insulin-like growth factor (IGF-1), platelet derived growth factor (PDGF), epidermal growth factor (EGF), transforming growth factor beta (TGF-β), basic fibroblast growth factor (bFGF), thymosin α1 (Tα1) and vascular endothelial growth factor (VEGF). More preferably, the agent is transforming growth factor beta (TGF-β) or other members of the TGF-β superfamily.

Additionally, agents that assist or stimulate healing may be added to a composition along with Tβ4 or a Tβ4 isoform. Such agents include angiogenic agents, growth factors, agents that direct differentiation of cells. For example, and not by way of limitation, Tβ4 or a Tβ4 isoform alone or in combination can be added in combination with any one or more of the following agents: VEGF, KGF, FGF, PDGF, TGFβ, IGF-1, IGF-2, IL-1, prothymosin α and thymosin α11 in an effective amount.

The actual dosage, formulation or composition that heals or prevents inflammation, damage and degeneration may depend on many factors, including the size and health of a subject. However, persons of ordinary skill in the art can use teachings describing the methods and techniques for determining clinical dosages as disclosed in PCT/US99/17282, supra, and the references cited therein, to determine the appropriate dosage to use.

In preferred embodiments, the concentration of the polypeptide is within a range of about 0.01-1 mole of the polypeptide per mole of the adhesive, more preferably within a range of about 0.1-0.5 mole of the polypeptide per mole of the adhesive, most preferably within a range of about 0.2-0.4 mole of the polypeptide per mole of the adhesive.

Suitable formulations may include Tβ4 or a Tβ4 isoform at a concentration within the range of about 0.001-10% by weight, within the range of about 0.01-0.1% by weight, or even about 0.05% by weight.

The invention includes use of antibodies which interact with Tβ4 peptide or functional fragments thereof. Antibodies which consists essentially of pooled monoclonal antibodies with different epitopic specificities, as well as distinct monoclonal antibody preparations are provided. Monoclonal antibodies are made from antigen containing fragments of the protein by methods well known to those skilled in the art as disclosed in PCT/US99/17282, supra The term antibody as used in this invention is meant to include monoclonal and polyclonal antibodies.

In yet another embodiment, the invention provides a method of treating a subject by administering an effective amount of an agent which modulates Tβ4 gene expression. The term “modulate” refers to inhibition or suppression of Tβ4 expression when Tβ4 is over expressed, and induction of expression when Tβ4 is under expressed. The term “effective amount” means that amount of Tβ4 agent which is effective in modulating Tβ4 gene expression resulting in effective treatment. An agent which modulates Tβ4 or Tβ4 isoform gene expression may be a polynucleotide for example. The polynucleotide may be an antisense, a triplex agent, or a ribozyme. For example, an antisense directed to the structural gene region or to the promoter region of Tβ4 may be utilized.

In another embodiment, the invention provides a method for utilizing compounds that modulate Tβ4 activity. Compounds that affect Tβ4 activity (e.g., antagonists and agonists) include peptides, peptidomimetics, polypeptides, chemical compounds, minerals such as zincs, and biological agents.

While not be bound to any particular theory, the present invention may promote healing or prevention of inflammation or damage by inducing terminal deoxynucleotidyl transferase (a non-template directed DNA polymerase), to decrease the levels of one or more inflammatory cytokines, or chemokines, and to act as a chemotactic factor for endothelial cells, and thereby promoting healing or preventing degenerative changes in tissue brought about by injury or other degenerative or environmental factors.

The invention is further illustrated by the following example, which is not to be construed as limiting.

### EXAMPLE

Proteins and Reagents

Human fibrinogen depleted of plasminogen, fibronectin and von Willebrand factor was purchased from Enzyme Research Laboratories (South Bend, Ind.). The recombinant αC-fragment corresponding to the human fibrinogen αC-domain (residues Aα221-610) and its truncated variants corresponding to the NH₂ and COOH-terminal halves (residues Aα221-391 and Aα392-610, respectively) were produced in E. coli using the pMT20b expression vector. The recombinant γ-module comprising residues 148-411 of the human fibrinogen γ chain was produced in E. coli using the same expression vector.

Bovine thrombin (1,000 NIHu/mg, aprotinin (4.4 TIU/mg), antirabbit IgG-horseradish conjugate and fluorescein isothiocyanate (FITC) were purchased from Sigma. Recombinant factor XIII was provided as a gift by Zymogenetics, Inc. (Seattle, Wash.). Synthetic thymosin β4 was provided as a gift by Regenerx Biopharmaceuticals, Inc. (Bethesda, Md.). Anti-thymosin β4 serum was prepared according to published methods.

Activation of Factor XIII

Factor XIII in 25 mM Tris buffer, pH 8.0, with 0.15 M NaCl (TBS), was activated either with thrombin or with CaCl₂; the latter was made to avoid the presence of thrombin which could potentially activate fibrinogen. Thrombin-activated FFXIII [FXIIIa(THr)] was made by addition of bovine thrombin to final concentrations of 25 NIH u/ml and 2.5 CaCl₂ mM. Ca²⁺-activated thrombin [FXIIIa(Ca)] was made by addition of CaCl₂ to final concentration of 50 mM. Final concentration of FXIII in both mixtures was 1.5 mg/ml; both mixture were incubated at room temperature for 10 min prior experiments.

Labeling of Thymosin β4 with FITC

Fluorescence labeled thymosin β4 was prepared by the reaction with fluorescein isothiocyanate (FITC). Thymosin β4 was transferred in 0.1 M NaHCO₃ buffer, pH 9.5, by gel-filtration on NAP5 Sephadex G-25 column (Amersham Biosciences) followed by addition of a 1.2 molar excess of FITC and incubation of the mixture at 37° C. for 2 h in the dark. Non-reacted FITC was removed on NAP5 column. The degree of labeling determined spectrophotometrically as described was found to be 0.9 mole of FITC per mole of thymosin β4.

Solid-Phase Binding Assay

The interaction between thymosin β4 and fibrin(ogen) and its fragments in the presence or absence of FXIIIa was studies by ELISA using plastic microliter plates. Wells of microliter plates were coated overnight at −4° C. with fibrinogen and fibrin at 10 μg/mL or with the recombinant fragments of 20 μg/ml, all in 0.1 M NaHCO₃ buffer, pH 8.3. Fibrin was made by addition to the wells of a mixture containing 10 μg/mL fibrinogen 1 NIH u/ml thrombin and 400 u/ml aprotinin, followed by overnight incubation at +4° C. The wells were then blocked by incubation with Super Blocker (Pierce) at 37° C. for 1 h. Following washing with TBS containing 0.05% Tween-20 (TBS-Tween), the indicated concentrations of thymosin β4, FXIII, FXIIIa(Thr) and FXIIIa(Ca) were added to the wells and incubated for 2-2.5 h at 37° C. Bound (incorporated) thymosin β4 was detected by the reaction with rabbit anti-thymosin β4 serum and peroxidase-conjugated anti-rabbit IgG. A TMB Microwell Peroxidase Substrase was added to the wells, and the incorporated thymosin β4 was measured spectrophotometrically at 450 nm. Incorporation of thymosin β4 into fibrinogen and fibrin

Reactions of incorporation of FITC-labeled and unlabeled thymosin β4 into fibrinogen and fibrin were performed in Eppendorf tubes containing a mixture of fibrinogen at 3 mg/mL (9 μM) and thymosin β4 or FITC-labeled thymosin β4 at 150 μg/L (30 μM) in 100 μL TBS with 2.5 mM CaCl₂. The reactions were initiated by addition of FXIIIa(Ca) or FXIIIa(Thr) to final concentration of 30 μg/mL. The final concentration of thrombin in the FXIIIa(Thr)-containing mixtures was made at 2.5 NIH u/mL, sufficient to rapidly form fibrin clot which was observed visually. The reactions with FITC-labeled thymosin β4 lasted for 4 hours at 37° C. in the dark and were stopped by heat inactiviation of the enzymes in boiling water for 5 min during fibrinogen and fibrin denatured and precipitated. The pellets were centrifuged and washed 3 times in TBS and then solubilized. The amounts of fibrin(ogen) and FITC-labeled thymosin β4 in the solubilized pellet were determined spectrophotomertrically using absorption molar coefficients E_{280.1%}=15.0 and ε₄₉₅=72,000 M⁻¹cm⁻¹, respectively. To prepare samples with unlabeled thymosin β4 for analysis by SDS-PAGE and Western blot the reaction mixtures at the indicated time were heat-inactivated as above and solubilized by addition of sample buffer (Invitrogen) containing SDS and reducing agent.

Kinetic Analysis

To analyze kinetics of the incorporation of thymosin β4 into different fibrin(ogen) fragments, they were immobilized onto the wells of microliter plates (as described above, except that the concentration of all fragments was 20 μg/mL) and incubated with several concentrations of thymosin β4 in the presence of 10 μg/L thrombin-activated factor XIIIa. The incubation mixtures were inhibited every 15 min during 1 hour of incubation by the addition of iodacetamide to final concentration 10 nM. Incorporated thymosin β4 at each time point was detected with rabbit anti-thymosin β4 serum as described above. The initial rates of the reaction of incorporation (V) at different concentrations of thymosin β4 were determined from the slopes of the reaction time course plots and expressed as tangent α=A₄₅₀/t (min), where A₄₅₀ represents absorbance at 450 nm in optical units (o.u) which is proportional to the amount of incorporated thymosin β4. Apparent Michaelis constants, Kₘ, were obtained from Lineweaver-Burk plots, 1/V (min/o.u.) versus 1/[S](μM⁻¹), where [S] is concentration of thymosin β4.

Western Blot Analysis

Detection of thymosin β4 incorporated into fibrin(ogen) and its fragments was performed as follows. The samples prepared as described above were electrophoresed and electrotransferred to a nitrocellulose membrane (Invitrogen) as described earlier. The membrane was blocked with a casein blocker for 1 hour and thymosin β4 was detected by the reaction with rabbit anti-thymosin β4 serum and peroxidase-conjugated anti-rabbit IgG. Visualization of the peroxidase-labeled protein bands was performed by the procedure recommended by the manufacturer using a supersignal west pico chemiluminescent substrate.

ELISA-Detected Incorporation of Thymosin β4 into Fibrinogen and Fibrin

To test that factor XIIIa could mediate cross-linking of thymosin β4 to fibrin(ogen), and to clarify the mechanism of such cross-linking we performed a direct study of the interaction of thymosin β4 with fibrinogen and fibrin in the presence and absence of recombinant factor XIII. It should be noted that the recombinant factor comprises two a subunits (a₂), in contrast to plasma factor XIII corresponds to the platelet form of factor XIII.

In ELISA experiments, when thymosin β4 at 150 μg/mL (30 μm) was incubated with immobilized fibrinogen, only a low signal was observed in the absence of factor XIII as well as in the presence of non-activated factor XIII suggesting that the interaction between them is very weak, if any. When thymosin β4 was incubated with immobilized fibrin in the absence or presence of non-activated factor XIIIa, which was activated by the addition of CaCl₂ to avoid conversion of fibrinogen into fibrin in the wells, the signal substantially increased suggesting that factor XIIIa mediates binding (incorporation) of thymosin β4 into fibrinogen. A similar situation was observed with immobilized fibrin except that the level of the incorporation was higher than that into fibrinogen. The incorporation in both cases was dose-dependent. The incorporation onto fibrin was further increased when factor XIII was activated with thrombin instead of Ca²⁺. Such differences could be due to different specific activities of these two factor XIIIa species. These results indicate that, activated XIII, similarly to tissue transglutaminase, mediates incorporation of thymosin β4 into both fibrinogen and fibrin. They also suggest that there is no significant non-covalent interaction thymosin β4 and both fibrinogen and fibrin.

Further Analysis of the Incorporation of Thymosin β4 into Fibrinogen and Fibrin

To further characterize factor XIIIa-mediated incorporation of thymosin β4 into fibrin(ogen), a mixture was analyzed of thrombin, factor XIII, thymosin β4 and fibrin at different time points by immunoblotting. The mixture and the samples for analysis were prepared as described in Experimental Procedures. The samples were electrotransferred to a nitrocellulose membrane and probed with anti-thymosin β4 serum. The results of immunobilizing indicate that factor XIIIa incorporates thymosin β4 into fibrin covalently, like tissue transglutaminase, and that the amount of the incorporated (cross-linked) thymosin β4 seems to reach saturation after 4 hours. This time was selected to evaluate the degree of the incorporation. For this purpose thymosin β4 was labeled with a FITC chromophore group which enabled its direct measurement in fibrinogen/thymosin β4 and fibrin/thymosin β4 mixtures. Such modification did not influence its incorporation into either fibrinogen or fibrin based on the pattern of incorporation revealed by Western blot analysis. A similar mixture as above but with FITC-labeled thymosin β4 was incubated for 4 hours after which the degree of incorporation was estimated base don the spectrophotometrically determined amounts of fibrin(ogen) and incorporated FITC-thymosin β4 in each sample. The results revealed that at the selected conditions, which include physiological concentration of fibrinogen (9 μM), factor XIIIa incorporated a substantial amount of FITC-thymosin β4, about 0.2 and 0.4 moles per mole of fibrinogen and fibrin, respectively.

Incorporation of Thymosin β4 into Individual Fibrin(ogen) Chains

To establish which of the three fibrin(ogen) chains are involved in cross-linking with thymosin β4, we analyzed the time course of factor XIIIa-mediated cross-linking of fibrinogen and fibrin in the presence and absence of thymosin β4 by SDS-PAGE and Western blot. It is well known that in fibrin factor XIIIa cross-links rapidly the COOH-terminal portions of the γ chains to produce γ-γ dimers followed by cross-linking of the α chains to form α-α dimers, trimers, and α-polymers; fibrinogen is cross-linked in a similar way but at a slower rate. When analyzed by SDS-APGE in reducing conditions, the bands corresponding to the individual polypeptide chains of fibrinogen and fibrin, Aα, Bβ, γ and α, β, γ, respectively, were well resolved. Incubation of fibrinogen with factor XIIIa resulted in progressive depletion of the band corresponding to the γ-γ dimers and the Aα-Aα dimers and trimers; the appearance of some material at the start which most probably corresponds to the Aα polymers was also observed. When fibrinogen was incubated with factor XIIIa in the presence of thymosin β4, no substantial difference in the intensity of the bands corresponding to the individual chains and their cross-linked variants was found. Similar results were obtained with fibrin except that the cross-linking of its α and γ chains occurred more rapidly, as expected, and the amount of the material at the start was higher. Subsequent Western blot experiments revealed that after 30 min of incubation substantial amount of thymosin β4 was incorporated into fibrinogen Aα chain and that after 150 min of incubation some thymosin β4 was also incorporated into the Aα-Aα dimer. The incorporation of thymosin β4 into fibrin α chain and the α-α dimer was much more rapid and after 150 min of incubation material of thymosin β4 was also observed in higher molecular mass forms of the α chain (α polymers). These results indicate that the fibrinogen Aα and fibrin α chains contain the major sites for covalent incorporation of thymosin β4. At the same time the appearance after 150 min of incubation of a low intensity band with the mobility between that of the γ-γ and α-α dimers suggests that thymosin β4 could also be incorporated into the fibrin γ chains (γ-γ dimer). Alternatively, this band may correspond to a proteolytically truncated variant of the α-α dimer.

Incorporation of Thymosin β4 into Recombinant Fibrin(ogen) Fragments

It is well established that the COOH-terminal proteins of the fibrinogen Aα and γ chains forming the αC domain and γ-module contain reactive Gln and Lys residues which are cross-linked by factor XIIIa in fibrin and therefore could potentially be involved in cross-linking with thymosin β4. To test this and to further localize the cross-linking sites for thymosin β4 in fibrin(ogen), was analyzed incorporation of thymosin β4 into the recombinant γ-module (residues γ148-411) and the αC-domain (Aα221-391 and Aα392-610 sub-fragments, by SDS-PAGE and Western blotting. Incubation of the αC-domain and the γ-module with factor XIIIa in the presence of thymosin β4 resulted in effective cross-linking and appearance of their appearance of their higher molecular mass forms, dimers, trimers and oligomers. At the same time, the cross-linking of the Aα221-391 and Aα392-610 sub-fragments, which contain mainly acceptor Gln and donor Lys residues, respectively, was much less effective. When the samples were electrotransferred to nitrocellulose membrane and probed with anti-thymosin β4 serum, substantial amounts of thymosin β4 were detected in the αC-domain, the γ-module and their higher molecular mass variants, dimers, trimers and oligomers. The incorporation into the Aα392-610 sub-fragment monomer and oligomers was also substantial while only very small amount of thymosin β4 was detected in the Aα221-391 oligomers. These results suggest that thymosin β4 could be cross-linked to both the αC-domain and the γ-module, and that the reactive Lys residues of the Aα392-610 region of the former are involved in the cross-linking.

The above observations were confirmed by ELISA. When thymosin β4 was incubated with the immobilized γ-module or the αC-domain variants in the presence of factor XIIIa, it was incorporated effectively into the γ-module and into the αC-domain and the Aα392-610 sub-fragment while the incorporation into Aα221-391 was very low. It should be noted that the incorporation of the γ-module was almost twice lower than that of the αC-domain variants at all concentration studied. When thymosin β4 was incubated with the same immobilized species in the presence of non-activated factor XIII or without it, the incorporation was very low in all cases. This suggests that, as in the case with fibrinogen and fibrin, there is no significant non-covalent interaction between thymosin β4 and the recombinant fragments.

It was previously shown that factor XIIIa cross-linking of the γ chains of fibrin exhibits apparent Michaelis behavior. Assuming that factor XIIIa behaves as a Michawlis enzyme when cross-linking thymosin β4 to the immobilized γ-module and αC-domain variants one could determine the kinetic parameters of such cross-linking. The analysis of the kinetic data revealed the following values of apparent Michaelis constants (Kₘ) for the reaction of incorporation, 183±29 μM for the incorporation of thymosin β4 into the γ-module, and 17.6±2.5 μM and 8.6±3.7 μM for that into the αC-domain and its Aα392-610 sub-fragment, respectively. The much higher Kₘ value for the γ-module than those for the αC-domain and its sub-fragment indicates that the cross-linking of thymosin β4 to the αC-domain variants is much more efficient. In this connection, the Kₘ for the Aα392-610 fragment is comparable to the Kₘ=6.2 μM determined previously for the factor XIIIa-mediated γ-γ cross-linking. The two-fold difference in the Kₘ values for the αC-domain and the Aα392-610 sub-fragment could be explained by competition between reactive Gln residues of thymosin β4 and the Aα392-610 region, i.e., between αC-to-αC and thymosin β4-to-αC cross-linking. In agreement, the double-reciprocal plot for the αC-domain and the Aα392-610 sub-fragment exhibits a pattern characteristic for competitive inhibition.

Altogether, the results indicted that factor XIIIa effectively cross-links thymosin β4 to the COOH-terminal portion of the isolated αC-domain including residues Aα392-610, that the incorporation into the isolated γ-module is less effective, and that in fibrinogen or fibrin the incorporation occurs mainly in the αC-domains.

Fibrin(ogen) plays an important role in wound healing through interactions with physiologically active proteins and cell receptors. Particularly, the fibrin matrix stimulates an inflammatory response and capillary tube formation by endothelial cells (angiogenesis), which are essential steps in the wound healing process, through interaction with the leukocyte integrin Mac-1 and endothelial cell receptor VE-cadherin, respectively. It also interacts with high affinity with basic fibroblast growth factor (bFGF) and vascular endothelial growth factor (VEGF) providing co-localization of these potent stimulators of angiogenesis at sites of fibrin deposition and their contribution to wound healing. Fibrin can also retain at insulin-like growth factor binding protein-3 (IGFPB-3), which forms a complex with IGF-1. Thymosin β₄, a potent angiogenic and wound healing factor, can also be incorporated into fibrin by tissue transglutaminase and apparently further increase the wound healing potential of fibrin matrix.

Although all transglutaminases catalyze the same reaction, formation of covalent γ-glutamyl-ε-lysyl isopeptide bonds between reactive Gln and Lys residues, their specificity towards substrates may differ. For example, while factor XIIIa, a plasma transglutaminase, specifically cross-links in fibrin the γ and α chains resulting in the γ-γ dimers and α-polymers, respectively, tissue transglutaminase is less specific and can also generate α-γ chains cross-links. The cross-linking patterns for the serine protease inhibitor (serpin), PAI-2, to fibrin(ogen) were also found to be different for tissue transglutaminase and factor XIIIa. It was originally shown that thymosin β₄ is incorporated into fibrin by guinea pig liver tissue transglutaminase; its incorporation into fibrin by factor XIIIa was hypothesized based on the facts that thrombin-activated platelets co-release factor XIII and thymosin β₄ and that the latter becomes cross-linked to fibrin. In this study it was demonstrated directly that thymosin β₄ is incorporated by factor XIIIa to both fibrinogen and fibrin. Furthermore, it was found that the degree of the incorporation is rather high, 0.2 and 0.4 mole of thymosin β₄ per mole of fibrinogen and fibrin, respectively. Since concentration of fibrinogen in plasma is about 9 μM, local concentration of fibrin at places of fibrin deposition should be much higher. Taking into account that thymosin β₄ exhibits its proangiogenic activity at 0.1 nM-1 μM, such degree of incorporation is obviously physiologically significant and should be sufficient to increase the wound healing potential of fibrin clot.

It is known that factor XIIIa incorporates into fibrin a number of plasma proteins, α₂-antiplasmin, PAI-2, fibronectin, thrombospondin, and von Willebrand factor. The mechanism of incorporation is established only for some of them. For example, fibronectin binds to the fibrin αC-domains non-covalently with high affinity prior to covalent cross-linking with factor XIIIa; the recognition sites and the reactive Gln and Lys residues in each protein are located in different regions providing proper orientation of the cross-linking sites. In addition, factor XIIIa interacts with the αC-domains further increasing the specificity of the reaction. To test whether non-covalent binding of thymosin β₄ precedes its cross-linking to fibrin, its interaction was studied with immobilized fibrinogen and fibrin in the presence and absence of non-activated factor XIII. In contrast to other proangiogenic factors such as bFGF and VEGF, which exhibit high affinity to fibrin, no noticeable non-covalent interaction was observed with thymosin β₄ in all cases. The incorporation was observed only in the presence of activated factor XIIIa suggesting that the covalent cross-linking may be the only mechanism to retain thymosin β₄ in fibrin clot.

The results clearly indicate that although thymosin β₄ could be incorporated by factor XIIIa into the isolated γ-module and the αC-domain variants, in fibrin(ogen) it is cross-linked mainly to the αC-domains, namely to their Aa392-610 regions. The analysis of distribution of the identified reactive Lys and Gln residues in thymosin β₄ and fibrin(ogen) provides a reasonable explanation for this finding. Thymosin β₄ contains a reactive amine donor, Lys38, and two amine receptors, Gln23 and Gln36, which could be involved in the cross-linking reaction with other proteins. There are only two reactive residues in the γ chain involved in the intermolecular γ-γ cross-linking, Gln398 (or Gln399) and Lys406, both located in the γ-module. When the isolated γ-module was treated with factor XIIIa, the cross-linking seemed to occur randomly resulting in dimers, trimers/oligomers; thymosin β₄ was incorporated in all these species. In fibrin, these regions are aligned by the DD:E interactions in an antiparallel manner facilitating cross-linking between Gln398/399 of one chain and Lys406 of another to form γ-γ dimers. The efficiency of this cross-linking reaction is much higher than that between these residues and thymosin β₄, and therefore it is not surprising that little or no incorporation of thymosin β₄ into the fibrin γ chains was observed in this study.

In contrast to the γ chain, the Aα chain contains multiple reactive glutamine and lysine residues. The following residues were found to be involved in the cross-linking between the α chains in fibrin or the recombinant αC-domains, Gln221, 237, 328 and 366, and Lys508, 539, 556, 580 and 601. The Aα chain Lys303 was shown to serve as amine donor in factor XIIIa-mediated cross-lining of the serpin α₂-antiplasmin to fibrin(ogen). This Lys is not reactive towards another serpin, PAI-2, which is cross-linked by tissue transglutaminase and factor XIIIa through other Aα chain lysine residues, 148, 176, 183, 230, 413 and 457. The study with a synthetic peptide mimicking the cross-linking region of α₂-antiplasmin revealed that it is incorporated into fibrin α chain through 12 reactive lysine residues, Lys418, 448, 508, 539, 556 and 580, which accounted for 78% of the total activity, and less reactive Lys208, Lys219 and/or 224, Lys427, 429, 601 and 606. At least 10 lysine residues within fibrin(ogen) Aα368-610 region were implicated in cross-linking reactions with fibronectin. The above analysis indicates that most of the identified reactive residues in fibrin are located in its αC-domains, that the 392-610 region of the αC-domain, to which thymosin β₄ is a preferentially cross-linked, contains at least 11 reactive Lys residues, and that among these residues only half is utilized in the α-α cross-linking. It also suggests that although thymosin β₄ could compete for reactive lysine residues involved in the α-α cross-linking, its cross-linking to the αC-domains may occur independently of their intermolecular α-α cross-linking providing its efficient incorporation into fibrin. Thus the reactive lysine residues of the αC-domains not only serve for the α-αcross-linking but also simultaneously accommodate physiologically active proteins, including thymosin β₄, which could modulate properties of fibrin matrix contributing to wound healing and other physiological and pathological processes.

Fibrinogen polymerizes in a controllable fashion to make a clot which easily adheres to different cells and is non-immunogenic and biodegradable. These make it an ideal hemostatic and bioadhesive (fibrin sealant) that has been used increasingly in numerous surgical applications as an hemostatic agent for the arrest of bleeding, and to assist tissue sealing and wound healing. The use of fibrin sealants in wound healing and other therapies can be enhanced by including bioactive agents. For example, it was shown in cellular and animal models that fibrin can serve as a vehicle for localized delivery of antibiotics and growth factors. While antibiotics encapsulated by fibrin are released slowly due to low solubility, the retention of growth factors in fibrin sealants was achieved through their high affinity interaction with fibrin, or through their direct covalent cross-linking to it. The ability of thymosin β₄ to be incorporated into fibrin(ogen) by cross-linking with factor XIIIa could be used for its immobilization on fibrin sealants. This study demonstrates high efficiency of such incorporation into both fibrinogen and fibrin, supporting this approach.

In summary, experimental studies confirm that thymosin β₄, a bioactive peptide, could be incorporated into fibrin by covalently cross-linking with factor XIIIa, demonstrated high efficiency of its incorporation into both fibrinogen and fibrin at physiological concentrations of the components, and localized the incorporation sites within the Aα392-610 region of the fibrin(ogen) αC-domains. Experimental data supports incorporation of physiologically significant amounts of thymosin β₄ into fibrin sealants for delivery to places of wound healing.

Tissue transglutaminase and presumably plasma transglutaminase, factor XIIIa, can covalently incorporate into fibrin(ogen) a physiologically active peptide, thymosin β₄. To clarify the mechanism of this incorporation interaction was studied of thymosin β₄ with fibrinogen, fibrin, and their recombinant fragments, the γ-module (γ chain residues 148-411), and the αC-domain (Aα chain residues 221-610) and its truncated variants by immunoblot and ELISA. No significant non-covalent interaction between them was detected in the absence of activated factor XIII while in its presence thymosin β₄ was effectively incorporated into fibrin and to a lesser extent into fibrinogen. The incorporation at physiological concentrations of fibrin(ogen) and factor XIII was significant with molar incorporation ratios of thymosin β₄ to fibrinogen and fibrin of 0.2 and 0.4, respectively. Further experiments revealed that although activated factor XIII incorporates thymosin β₄ into the isolated γ-module and αC-domain, in fibrin the latter serves as the major incorporation site. This site was further localized to the COOH-terminal portion of the αC-domain including residues 392-610.

1. A composition comprising a substantially purified composition including an adhesive and a polypeptide comprising amino acid sequence LKKTET SEQ ID NO: 1 or a conservative variant thereof.

2. The composition of claim 1 wherein said adhesive is capable of adhering to tissue of a living subject.

3. The composition of claim 2 wherein said adhesive is biodegradable.

4. The composition of claim 1 wherein said adhesive is fibrin, fibrinogen, fibrin glue, collagen, a fragment thereof, or a mixture thereof.

5. The composition of claim 4 wherein said adhesive and said polypeptide are covalently bound together.

6. The composition of claim 5 wherein said adhesive and said polypeptide are covalently bound by factor XIIIa.

7. The composition of claim 6 wherein said adhesive is a fragment of fibrin or fibrinogen.

8. The composition of claim 1 wherein said polypeptide comprises amino acid sequence KLKKTET SEQ ID NO:2 or LKKTETQ SEQ ID NO:3, Thymosin β4 (Tβ4), an N-terminal variant of Tβ4, a C-terminal variant of Tβ4, an isoform of Tβ4, a splice-variant of Tβ4, oxidized Tβ4, Tβ4 sulfoxide, lymphoid Tβ4 or pegylated Tβ4.

9. The composition of claim 1 wherein said polypeptide is recombinant or synthetic.

10. The composition of claim 1 wherein said polypeptide is an antibody.

11. The composition of claim 10 wherein said antibody is polyclonal or monoclonal.

12. The composition of claim 4 wherein the concentration of said polypeptide is within a range of about 0.01-1 mole said polypeptide per mole of said adhesive.

13. The composition of claim 12 wherein said range is about 0.1-0.5 mole said polypeptide per mole of said adhesive.

14. The composition of claim 13 wherein said range is about 0.2-0.4 mole said polypeptide per mole of said adhesive.

15. The method of delivering a polypeptide to a site, comprising introducing the composition of claim 1 to said site.

16. The method of claim 15 wherein said composition is applied to said site by spraying.

17. The method of claim 16 wherein said site is a wound.

18. The method of claim 15 wherein said adhesive is capable of adhering to tissue of a living subject.

19. The method of claim 18 wherein said adhesive is biodegradable.

20. The method of claim 15 wherein said adhesive is fibrin, fibrinogen, fibrin glue, collagen, a fragment thereof or a mixture thereof.

21. The method of claim 20 wherein said adhesive is covalently bound to said polypeptide.

22. The method of claim 21 wherein said adhesive is covalently bound to said polypeptide by factor XIIIa.

23. The method of claim 22 wherein said adhesive is a fragment of fibrin or fibrinogen.

24. The method of claim 15 polypeptide comprises amino acid sequence KLKKTET SEQ ID NO:2 or LKKTETQ SEQ ID NO:3, Thymosin β4 (Tβ4), an N-terminal variant of Tβ4, a C-terminal variant of Tβ4, an isoform of Tβ4, a splice-variant of Tβ4, oxidized Tβ4, Tβ4 sulfoxide, lymphoid Tβ4 or pegylated Tβ4.

25. The method of claim 15 wherein said polypeptide is recombinant or synthetic.

26. The method of claim 15 wherein said polypeptide is an antibody.

27. The method of claim 26 wherein said antibody is polyclonal or monoclonal.

28. The method of claim 20 wherein said polypeptide is a concentration that is within a range of about 0.1-1 mole said polypeptide per mole of said adhesive.

29. The method of claim 28 wherein said range is about 0.1-0.5 mole said polypeptide per mole of said adhesive.

30. The method of claim 29 wherein said range is about 0.2-0.4 mole said polypeptide per mole of said adhesive.
